(19) United States
(12) Patent Application Publication
(10) Pub. No.: US 2009/0054510 A1
(43) Pub. Date: Feb. 26, 2009

(54) METHOD FOR IDENTIFYING COMPOUNDS FOR THE TREATMENT OF DEPRESSION

(75) Inventors: Tine Bryan Stensbol, Vaerlose (DK); Arne Mork, Malov (DK)

Correspondence Address: DARBY & DARBY P.C., P.O. BOX 770, Church Street Station, New York, NY, 10008-0770, US

(73) Assignee: H. LUNDBECK A/S, Valby (DK)

(21) Appl. No.: 12/280,363

(22) PCT Filed: Mar. 21, 2007
(86) PCT No.: PCT/DK07/50036

§ 371 Date: Sep. 19, 2008

Related U.S. Application Data
(60) Provisional application No. 60/743,637, filed on Mar. 22, 2006.

(30) Foreign Application Priority Data
Mar. 22, 2006 (DK) PA 200600406

(51) Int. Cl. A61K 31/404; G01N 33/53

(52) U.S. Cl. 514/415; 435/7.21

(57) ABSTRACT

An assay for identifying compounds with increased efficacy in the treatment of depression is disclosed. The assay comprises the steps of determining whether a test compound inhibits SERT and whether it inhibits with an allosteric site ion SERT.

### FIELD OF THE INVENTION

The present invention relates to a method for the identification of compounds useful in the treatment of depression.

### BACKGROUND

The serotonin transporter (SERT) is probably the most important target for drugs for the treatment of mood disorders, such as depression and anxiety. Some drugs specifically inhibit SERT, whereas others have additional pharmacological activities, e.g. an agonistic or antagonistic effect on serotonin receptors. Specific SERT inhibitors are often referred to as SSRI, and drugs with a broader pharmacological profile are exemplified by the tricyclics. SSRI exert their effect by blocking the reuptake of serotonin, which again gives rise to an increase in the extra-cellular serotonin.

It has been known for some time now that different SERT inhibitors may bind to and block different sites on SERT. More recently, it has also been found that a compound such as (S)-citalopram binds to more than one site, i.e. a primary site and an allosteric site [J. Neurochem, 92, 21-28, 2005], and that the superior effects of (S)-citalopram may be attributed to this allosteric binding.

### SUMMARY OF THE INVENTION

The present inventors have surprisingly found that compounds which bind to an allosteric site of the serotonin transporter (SERT) and inhibit serotonin re-uptake, i.e. bind to the primary site of SERT are particularly useful in the treatment of depression. Consequently, in one embodiment, the invention provides a method for identifying compounds useful in the treatment of depression, the method comprising the steps of

a) Contacting the serotonin transporter with one or more concentration of a test compound;

b) Determining whether said test compound inhibits serotonin re-uptake;

c) Determining whether said test compound interacts with one or more allosteric site at the serotonin transporter; and

d) Selecting the test compound which inhibits serotonin re-uptake and which interacts positively with one or more allosteric site at the serotonin transporter;

wherein steps b) and c) may be taken in any order.

In another embodiment, the invention provides a method of treating depression comprising the administration of a therapeutically effective amount of a compound identified by a method of the present invention to a patient in need thereof.

In another embodiment, the invention provides the use of a compound identified by the method of the present invention in the manufacture of a medicament for the treatment of depression.

In another embodiment, the invention relates to a compound identified by the method of the present invention for use in the treatment of depression.

In another embodiment, the invention provides a pharmaceutical composition comprising a compound identified by the method of the present invention.

In another embodiment, the invention relates to a method for promoting the sales of an antidepressant, said method comprising the public distribution of information describing that the effects of said antidepressant is fully or partially attributable to the binding of said antidepressant to an allosteric site to which [2-(6-Fluoro-1H-indol-3-ylsulfanyl)-benzyl]-methyl-amine binds.

### FIGURES

FIGS. 1a and 1b: ³H-(S)-citalopram dissociation from SERT with [2-(6-Fluoro-1H-indol-3-ylsulfanyl) -benzyl]-methyl-amine.

FIG. 2: Association of [2-(6-Fluoro-1H-indol-3-ylsulfanyl)-benzyl]-methyl-amine and (S)-citalopram to SERT in the presence of different concentration of (R)-citalopram.

FIG. 3: Extra cellular serotonin levels (ventral hippocampus) in rats as measured by acute microdialysis after administration of [2-(6-Fluoro-1H-indol-3-ylsulfanyl) -benzyl]-methyl-amine±(R)-citalopram.

FIG. 4: Extra cellular serotonin levels (ventral hippocampus) in rats as measured by 3-days microdialysis after administration of [2-(6-Fluoro-1H-indol-3-ylsulfanyl) -benzyl]-methyl-amine or (S)-citalopram.

### DETAILED DESCRIPTION OF THE INVENTION

In one embodiment, the method of the present invention is used for the identification of compounds having improved efficacy in the treatment of depression. It is well-known that treatment of affective disorders, such as e.g. depression with classic selective serotonin re-uptake inhibitors (SSRI), such as e.g. fluoxetine, is associated with a lag-phase of up to several weeks before the medication starts to work. A generally accepted hypothesis to explain the lag-phase is that the onset of a therapeutic effect of the SSRIs is slow due to a slow elevation of 5-HT (serotonin) to therapeutic levels. This is believed to be due to a slow desensitization of 5-HT negative autoreceptors, specifically the somatodentritic 5-HT_{1A} autoreceptors (in the raphé nuclei) and terminal 5-HT_{1B} autoreceptors that restrain the release of 5-HT from the presynaptic nerve terminal.

In one embodiment, “increased efficacy” is intended to indicate that the therapeutic effect is improved when measured at different times following the start of the treatment. Therapeutic effect is typically measured using clinically relevant scales, such as e.g. MADRAS or HAM-D. Typically, the effect is measured 1 week after, 2 weeks after, 3 weeks after, 4 weeks after, 5 weeks after or 6 weeks after the start of the treatment. An improvement of the efficacy may be reflected in faster on-set of action, as increased clinical response after prolonged treatment, lower fraction of non-responders, or as a combination of these effects.

In one embodiment, the method of the present invention is used for the identification of compounds, which are superior to existing treatment, such as e.g. fluoxetine.

The method of the present invention comprises the determination of whether a test compound inhibits the re-uptake of serotonin, i.e. whether a test compound binds to the primary site of the serotonin transporter. Binding of a test compound to the serotonin transporter may be quatified by determining the IC₅₀ concentration, which is the concentration of the test compound giving rise to a 50% inhibition of the serotonin transport by the serotonin transporter. This value may be determined e.g. as described in example 1 herein. In one embodiment, test compounds selected according to the method of the present invention have IC₅₀ below 100 nM, such as below 50 nM, such as below 20 nM, such as below 10 nM, or even below 2 nM.

The method of the present invention comprises the determination of whether a test compound interacts with one or more allosteric site on the serotonin transporter. An allosteric site is a site on a protein, such as an enzyme, a receptor or a transporter, which is different from the primary or active site, but where the binding of a ligand to said allosteric site affects the binding of ligands to the active site. Binding of a ligand to an allosteric site may affect the binding of the same ligand or of another ligand to the primary site.

In the method of the present invention, a test compound is selected if it interacts positively with one or more allosteric site on the serotonin transporter. In the present context, “to interact positively” is intended to indicate that the test compound gives rise to a stabilisation of the binding between serotonin transporter primary site and a primary site ligand, e.g. the test compound itself. A stabilisation of said binding may be quantified by measuring the dissociation rates for the test compound:serotonin transporter primary site complex. A test compound is said to positively interact with an allosteric site if binding of said compound to said allosteric site gives rise to a decrease in the dissociation rate. In particular, the dissociation rates are decreased more in the presence of high concentration of a test compound than in low concentrations. Dissociation rates are measured at a broad range of concentrations of the test compound, such as e.g. 1 nM, 10 nM, 100 nM, 100000 nM, or 1000000 nM. Determination of dissociation rates can be made as described in Example 2 herein. In one embodiment, a compound is selected if it gives rise to a more than 2 fold slowing of the (S)-citalopram dissociation rate, such as more than a 5 fold slowing, such as more than a 10 fold slowing, such as more than a 100 fold slowing, measured at relevant concentrations, such as e.g. up to 100 μM, up to 200 μM or up to 500 μM.

Alternatively, a stabilisation of said binding may be quantified by measuring the association rates for the test compound:serotonin transporter primary site complex. A test compound is said to positively interact with an allosteric site if binding of said compound to said allosteric site gives rise to an increase in the association rate. Determination of association rates can be made as shown in example 7

In one embodiment, the method of the present invention comprises the further step of determining whether a test compound gives rise to an increase in the extra cellular serotonin level in the ventral hippocampus or prefrontal cortex of rats, and selecting compounds giving rise to such increase. A determination of whether the extra cellular serotonin level increases is made after the start of administration of a test compound. In particular, the determination can be made 3, 4, 5, 6, 7 or 14 days after start of the administration of the test compound, or it can be made acutely. In one embodiment, a test compound is selected if it gives rise to more than 300%, such as more than 400%, such as more than 500% increase in the extra cellular serotonin levels as measured acutely. In one embodiment, a test compound is selected if it gives rise to an increase of 300% or more in the extra cellular serotonin 3 days after start of the administration of the test compound. The determination of extra cellular serotonin levels in the ventral hippocampus or prefrontal cortex of rats can be made e.g. in a microdialysis experiments as described in Example 5 herein.

In one embodiment, the method of the present invention comprises the further step of determining whether an allosteric site with which a test compound interacts is the allosteric site of the serotonin transporter capable of binding R-citalopram. In one embodiment, test compounds are not selected if they interact positively only and solely with the allosteric site capable of binding (R)-citalopram. This determination can be made e.g. as described in example 3 herein.

(R)-citalopram and (S)-citalopram are ligands to the same allosteric site on the serotonin transporter. The binding of the two compounds to said allosteric site, however, gives rise to different effects. Binding of (S)-citalopram effects a strengthening of the (S)-citalopram:serotonin primary site complex, i.e. it increases the inhibition of serotonin reuptake. In contrast hereto, binding of (R)-citalopram to the allosteric site only brings about a smaller change in the binding of primary site ligands, such as (S)-citalopram to the primary site of the serotonin transporter. It is believed that this explains the superiority of (S)-citalopram over the racemat citalopram at even dosages [Psychopharm, 174, 163-176, 2004].

The data presented in example 6 show that [2-(6-Fluoro-1H-indol-3-ylsulfanyl)-benzyl]-methyl-amine binds to an allosteric site at SERT. Moreover, the data presented in example 7 shows that this allosteric site is different to that of (R)-citalopram, i.e. that the two compounds bind to different allosteric sites. This notion is supported by the in vivo results of example 8 wherein it is shown that (R)-citalopram does not influence the increase in serotonin levels brought about by [2-(6-Fluoro-1H-indol-3-ylsulfanyl)-benzyl]-methyl-amine. Keeping in mind that the only pharmacologically relevant binding of (R)-citalopram is to an allosteric site of SERT, the only way to fairly interpret the data of example 8 is that [2-(6-Fluoro-1H-indol-3-ylsulfanyl)-benzyl]-methyl-amine and (R)-citalopram binds to different allosteric sites. As shown in example 9, compounds binding to the allosteric site of [2-(6-Fluoro-1H-indol-3-ylsulfanyl)-benzyl]-methyl-amine, in casu the compound itself, produce a more prominent increase in the serotonin levels than compounds that do not, such as (S)-citalopram. As increase in serotonin levels is the basis for the clinical effect of serotonin reuptake inhibitors, it is expected that this difference will translate into clinical superiority of compounds binding to the allosteric site of [2-(6-Fluoro-1H-indol-3-ylsulfanyl)-benzyl]-methyl-amine.

In one embodiment, the method of the present invention comprises the step of determining whether the test compound interacts positively with the allosteric site of the serotonin transporter capable of binding [2-(6-Fluoro-1H-indol-3-ylsulfanyl)-benzyl]-methyl-amine, and selecting compounds that do. This determination can be made e.g. as described in example 4 herein.

The compounds of the present invention are useful for the treatment of depression. Hence, in one embodiment, the present invention relates to a method of treating depression which method comprises the administration of a therapeutically effective amount of a compound identified by a method of the present invention to a patient in need thereof, provided said compound is not [2-(6-Fluoro-1H-indol-3-ylsulfanyl)-benzyl]-methyl-amine.

A “therapeutically effective amount” of a compound as used herein means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspects of the invention. The patient to be treated is preferably a mammal, in particular a human being.

In one embodiment, the present invention relates to the use of a compound identified by the method of the present invention in the manufacture of a medicament for the treatment of depression, provided said compound is not [2-(6-Fluoro-1H-indol-3-ylsulfanyl)-benzyl]-methyl-amine.

In one embodiment, the invention relates to a compound identified by the method of the present invention for use as an antidepressant, provided said compound is not [2-(6-Fluoro-1H-indol-3-ylsulfanyl)-benzyl]-methyl-amine.

In one embodiment, the invention provides a pharmaceutical composition comprising a compound identified be the method of the present invention, provided said compound is not [2-(6-Fluoro-1H-indol-3-ylsulfanyl)-benzyl]-methyl-amine.

In one embodiment, the invention relates to a method for promoting the sales of an antidepressant, said method comprising the public distribution of information describing that the effects of said antidepressant is fully or partially attributable to the binding of said antidepressant to an allosteric site on the serotonin transporter to which [2-(6-Fluoro-1H-indol-3-ylsulfanyl)-benzyl]-methyl-amine binds. In particular, said antidepressant is an inhibitor of the serotonin transporter.

In one embodiment, the invention relates to a method for promoting the sales of an antidepressant which binds to the allosteric site of the serotonin transporter capable of binding [2-(6-Fluoro-1H-indol-3-ylsulfanyl)-benzyl]-methyl-amine, said method comprising the public spreading of information that the effects of said antidepressant is fully or partially attributable to its binding to an allosteric site, in particular an allosteric site on the serotonin transporter. In particular, said antidepressant is an inhibitor of the serotonin transporter.

In one embodiment, distribution of said information is achieved by a method selected from the group consisting of verbal communication, pamphlet distribution, print media, audio tapes, magnetic media, digital media, audiovisual media, billboards, advertising, newspapers, magazines, direct mailings, radio, television, electronic mail, braille, electronic media, banner ads, fiber optics, and laser light shows.

### EXAMPLE 1

### Inhibition of 5-HT Uptake in Rat Synaptosomes

Rat forebrain from male animals (125 g-225 g) is weighed out and homogenized with cooled buffer with a glas/teflon homogenizer in about 10 ml 0.40 M sucrose buffer containing 1 mM Nialamid, see below. The suspension is centrifuged for 10 min at 1000 G/4° C. The resulting supernatant is centrifuged for 20 min. at 40.000 G/4° C. The pellet is resuspended in 180× volumes KRP-buffer. The suspension is kept on ice until use. Aliquots consisting of 10 μl test compound, 10 μl 10.0 nM [³H]-5HT and 180 μl membrane (0.5 mg/well), resulting in a total volume of 200 μL are incubated for 15 minutes at 37° C. Non-specific binding/uptake is determined in the presence of 10 μM citalopram and the total uptake is determined in the presence of buffer. After the incubation bound ligand was separated from unbound by filtration through Unifilter GF/C, presoaked in 0.1% PEI for 30 minutes, using a Tomtec CellHarvester program (Uptake). The filters are washed once with 1 ml ice-cold buffer, dried at 50° C., and 35 μl scintillation liquid is added to the filters. Bound radioactivity is counted in a Wallac MicroBeta.

### EXAMPLE 2

### Dissociation Rates

Dissociation rates for the complex formed between the test compound and serotonin transporter bound at the primary site are determined on membrane preparations from HEK293 cells transiently expressing hSERT. The kinetics of the dissociation of the compound- SERT complex is determined after extensive dissolution (300×) of aliquots consisting of 10 μg membranes from HEK293 cells transiently expressing hSERT and 10 μL [³H]test compound pre-incubated until equilibrium. The concentration of [³H]test compound is approximately 6 times Kd. Dissociation is initiated by extensive dissolution (300×) of aliquots in the presence or absence of unradiolabelled test compound (0-300 μM). Bound [³H]test compound is measured after 0, 2, 5, 10, 15, 20, 30, 50, 70 and 90 minutes.

### EXAMPLE 3

### R-Citalopram

Association rates are determined on membrane preparations from HEK293 cells transiently expressing hSERT. The kinetics of the association of the [³H]compound- SERT complex is determined after incubation of aliquots consisting of 10 μg membranes from HEK293 cells transiently expressing hSERT and 290 μL [³H]test compound pre incubated until equilibrium. Association is initiated by addition of radioligand in the absence or presence of R-citalopram (0-300 μM). Bound [³H]test compound is measured after 0, 2, 5, 10, 15, 20, 30, 50, 70 and 90 minutes. [³H]Test compounds that are not affected by the addition of R-citalopram (0-300 μM) are selected.

### EXAMPLE 4

### Interaction at the [2-(6-Fluoro-1H-indol-3-ylsulfanyl)-benzyl]-methyl-amine Allosteric Site

The determination is made essentially as described in Example 3 above using [³H] [2-(6-Fluoro-1H-indol-3-ylsulfanyl)-benzyl]-methyl-amine.

### EXAMPLE 5

### Microdialysis

### Animals

Male Sprague-Dawley rats, initially weighing 275-300 g, are used

### Surgery and Microdialysis Experiments

Rats are anaesthetised with hypnorm/dormicum (2 ml/kg) and intracerebral guide cannulas (CMA/12) are stereotaxically implanted into the hippocampus, aiming to position the dialysis probe tip in the ventral hippocampus (co-ordinates: 5.6 mm posterior to bregma, lateral −5.0 mm, 7.0 mm ventral to dura or in the frontal cortex (co-ordinates: 3.2 mm anterior to bregma; lateral, 0.8 mm; 4.0 mm ventral to dura). Anchor screws and acrylic cement are used for fixation of the guide cannulas. The body temperature of the animals is monitored by rectal probe and maintained at 37° C. The rats are allowed to recover from surgery for 2 days, housed singly in cages. On the day of the experiment, a microdialysis probe (CMA/12, 0.5 mm diameter, 3 mm length) is inserted through the guide cannula.

The probes are connected via a dual channel swivel to a microinjection pump. Perfusion of the microdialysis probe with filtered Ringer solution (145 mm NaCl, 3 mM KCl, 1 mM MgCl₂, 1.2 mM CaCl₂) is begun shortly before insertion of the probe into the brain and continued for the duration of the experiment at a constant flow rate of 1 μL/min. After 180 min of stabilisation, the experiments are initiated. Dialysates were collected every 20 min.

Acute treatment; single injections:

Four fractions are sampled to determine the basal levels of extra cellular 5-HT. Thereafter, the test compound is administered and the sampling is continued for approx. 3 hours.

Data presentation. In single injection experiments the mean value of 3 consecutive 5-HT samples immediately preceding compound administration serves as the basal level for each experiment and data are converted to percentage of basal (mean basal pre-injection values normalized to 100%).

### Three-Day Treatment; Minipumps:

For three-day treatment experiments, osmotic minipumps (Alzet, 2ML1) are used. The pumps are filled with vehicle/test compound under aseptic conditions and implanted subcutaneously under anaesthesia. The experiments are carried out with the minipumps on board. Blood samples for measuring plasma levels of compounds after 3 days of treatment are collected at the end of the experiments.

Data presentation. In minipump experiments mean of baseline 5-HT samples from treated animals are compared to mean of baseline 5-HT samples from vehicle animals; normalizing the vehicle group to 100%.

After the experiments the animals are sacrificed, their brains removed, frozen and sliced for probe placement verification.

### Analysis of Dialysate 5-HT

Concentration of 5-HT in the dialysates is analysed by means of HPLC with electrochemical detection. The 5-HT is separated by reverse phase liquid chromatography (ODS 150×3 mm, 3 uM). 5-HT: Mobile phase consisting of 75 mM NaH₂PO₄, 150 ml/l sodium octanesulfonic acid, 100 ul/l triethylamine and 10% acetonitrile (pH 3.0) at a flow rate of 0.4 ml/min. Electrochemical detection was accomplished using a coulometric detector; potential set at 250 mV (guard cell at 350 mV) (Coulochem II, ESA).

### EXAMPLE 6

### Binding of [2-(6-Fluoro-1H-indol-3-ylsulfanyl)-benzyl]-methyl-amine to an Allosteric Site on SERT

The assay procedure was modified based on the methods described in Pharmacol. Toxicol., 80, 197-201, 1997 and J.Neurochem., 92, 21-28, 2005.

PeakRapid 293 cells (Edge Biosystems, Gaithersburg, Md.) were cultured in Dulbecco's Modified Eagle Medium (DMEM) supplemented with 10% bovine calf serum, 4 mM L-glutamine, 100 units/ml penicillin and 100 μg/ml streptomycin) at 37° C., 5% CO2. Human SERT cDNA in a mammalian expression vector was transiently transfected into PeakRapid 293 cells using 10 μg total DNA in 150 cm2 flasks by lipofectamine method (Invitrogen, Carlsbad, Calif.). Two days after transfection, membranes were harvested.

Binding assays were conducted in buffer containing 50 mM Tris, 120 mM NaCl, and 5 mM KCl, PH 7.4. Prior to dissociation binding, [3H]-(S)-citalopram (40 nM) was incubated with hSERT membranes (5 ug/10 ul) for 2 hours at room temperature. The reaction mixture was dispensed into 96 well format minitubes at 10 ul/well and kept on ice. Dissociation binding was carried out at room temperature and was started at different time points by adding 800 ul of buffer or the test compound at various concentrations. Assay was terminated by rapid filtration using a Brandel harvester followed by washing 3 times with ice-cold wash buffer (50 mM Tris, PH 7.4). Bound ligand was counted using a Trilux counter.

Dissociation binding data were analyzed using GraphPad Prism 4 (GraphPad, San Diego, Calif.) and Xlfit 4 (IDBS, Guildford, Surrey, UK). A one-phase exponential decay equation was used to fit binding values at different time points, giving dissociation half-life (t½) and rate (k) values. The ratios (k/k₀) of the rate (k) in the presence compounds over the rate (k₀) in the absence of compounds were calculated for each compound concentration. IC₅₀ values were obtained by fitting k/k₀ and log concentrations in sigmoidal dose response curves. The maximal k/k₀ (fold modulation) at the highest concentration was also reported. Results are averaged from N=2 assays.

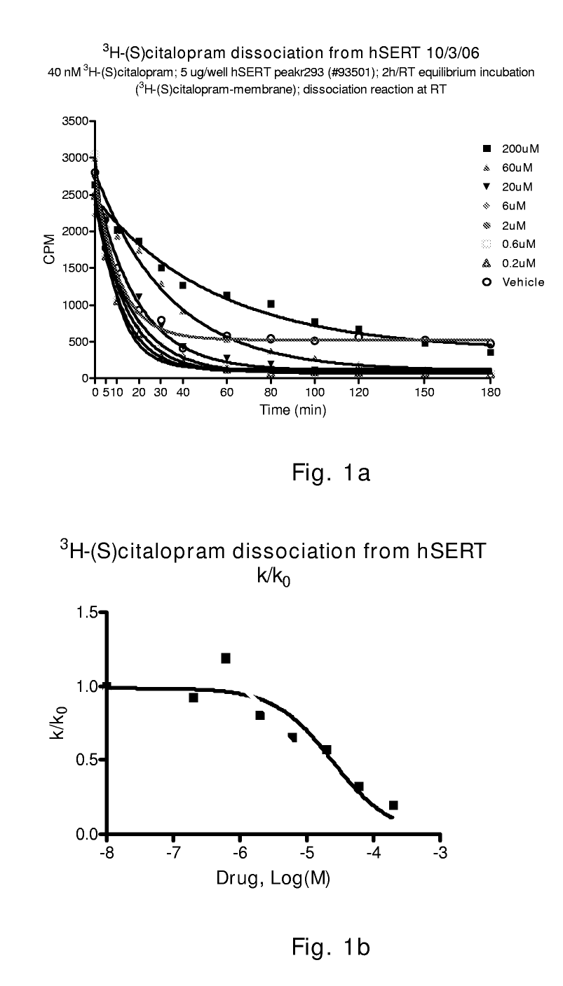

The data obtained are depicted in FIG. 1a and 1b, and 2-(6-Fluoro-1H-indol-3-ylsulfanyl) -benzyl]-methyl-amine binds to an allosteric site with an IC₅₀ of 26 μM and slowed 3H (S)-citalopram by a rate of 5.4 fold at 200 μM.

### EXAMPLE 7

Association of [2-(6-Fluoro-1H-indol-3-ylsulfanyl)-benzyl]-methyl-amine and (S)-Citalopram to SERT in the Presence of (R)-Citalopram

HEK293 cells expressing hSERT are harvested and 10 μg HEK293-hSERT membrane is placed in each well in a 96 well microtiter plate. Association is started by adding 290 μL [3H](S)citalopram+test-compounds. The final volumen is 300 μL and the concentration of [3H](S)citalopram is approximately 6 nM. Concentration of test-compounds is varied from the nM to the μM range. Incubation-time is 0, 2, 5, 10, 15, 20, 30, 50, 70 and 90 minutes on ice or at room temperature (22° C.).
Mebranes are harvested in ice-cold ELGA-water onto a GF/C fiberglass filter (Presoaked with 0.5% PEI for 30 min). Filters are rinsed twice with 2×0.5 ml ice-cold ELGA-water. The filters are dried and added 40 L of scintillation fluid. After 2 hours, the bound [3H](S)citalopram is measured in a scintillation counter.

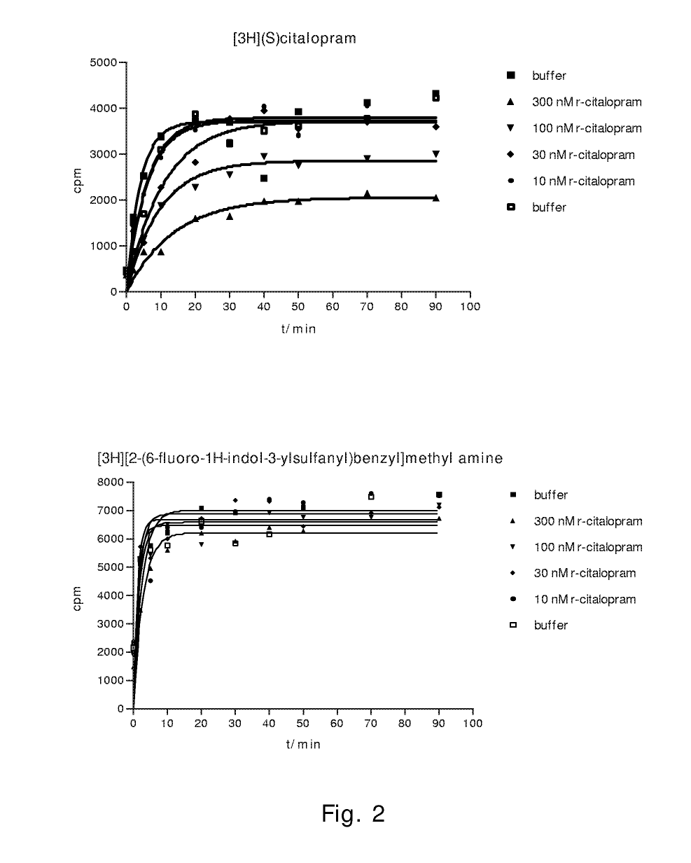

The data obtained are depicted in FIG. 2. The data clearly indicate that [³H] (S)-citalopram and [³H] 2-(6-Fluoro-1H-indol-3-ylsulfanyl)-benzyl]-methyl-amine show dissimilar binding properties to SERT. Association of [³H] (S)-citalopram to SERT is clearly affected by (R)-citalopram, decreasing the association with increasing concentrations of (R)-citalopram, whereas association of [³H] [2-(6-Fluoro-1H-indol-3-ylsulfanyl)-benzyl]-methyl-amine was almost unaffected by (R)-citalopram in the same concentration range. This leads to the conclusion that (S)-citalopram and [2-(6-Fluoro-1H-indol-3-ylsulfanyl)-benzyl]-methyl-amine bind to different allosteric sites.

### EXAMPLE 8

### Subcronic Microdialysis in Rats

### The Experimental Details are Essentially as Described in Example 5

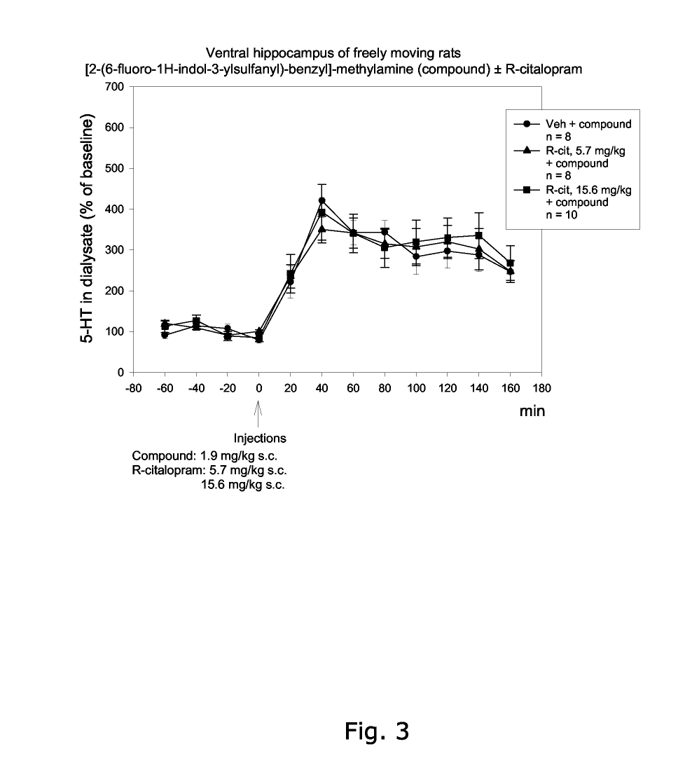

The data obtained are shown in FIG. 3. It is clearly shown that [2-(6-fluoro-1H-indol-3-ylsulfanyl) -benzyl]-methyl-amine gives rise to a significant increase in the extracellular serotonin in the ventral hippocampus. Moreover, this increase is not affected by concomitant administration of (R)-citalopram. This leads to the conclusion that (R)-citalopram and [2-(6-Fluoro-1H-indol-3-ylsulfanyl)-benzyl]-methyl-amine do not compete for the same binding site, i.e. that the two compounds bind to different allosteric sites. These data confirm the conclusions from Example 7 in an in vivo setting.

### EXAMPLE 9

### 3-Day Microdialysis in Rats

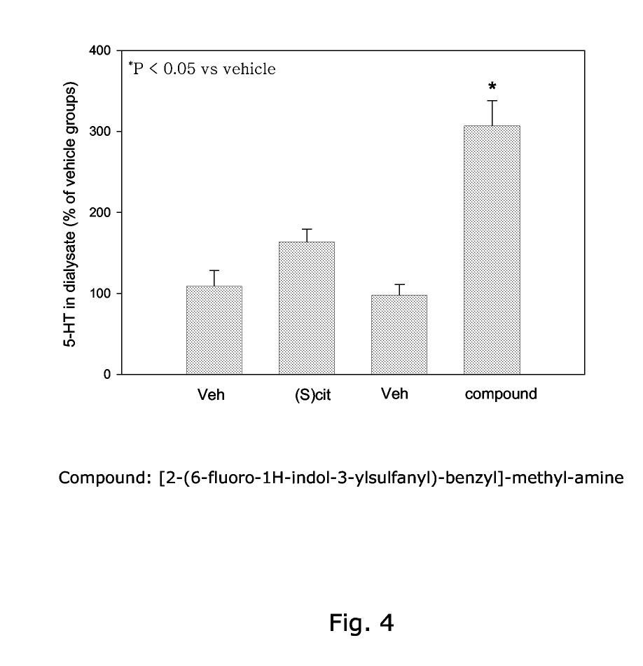

See example 5 for experimental data. The compounds were dosed as follows: 10 mg/kg (S)-citalopram and 4 mg/kg [2-(6-Fluoro-1H-indol-3-ylsulfanyl)-benzyl]-methyl-amine; both as the oxalate salt.
The data presented in FIG. 4 show that 2-(6-Fluoro-1H-indol-3-ylsulfanyl)-benzyl]-methyl-amine gives rise to a faster and more pronounced increase in the serotonin levels than (S)-citalopram. It is expected that these finding will translate into improved efficacy in a clinical setting.

1. A method for identifying compounds useful in the treatment of affective disorders, the method comprising the steps of
a) Contacting the serotonin transporter with one or more concentration of a test compound;
b) Determining whether said test compound inhibits serotonin re-uptake;
c) Determining whether said test compound interacts with one or more allosteric site at the serotonin transporter; and
d) Selecting the test compound which inhibits serotonin re-uptake and which interacts positively with one or more allosteric site at the serotonin transporter;
wherein steps b) and c) may be taken in any order.

2. The method according to claim 1 for the identification of compounds which are superior to fluoxetin® in the treatment of depression.

3. The method according to claim 1, wherein a test compound is selected in step d) if it has an IC₅₀ for the inhibition of the serotonin transporter below 100 nM.

4. The method according to claim 1, wherein said method comprises the further step of determining whether an allosteric site with which a test compound interacts is the allosteric site of the serotonin transporter capable of binding R-citalopram, and wherein a test compound positively interacting with said site is not selected.

5. The method according to claim 1, wherein said method comprises the further step of determining whether the extra cellular serotonin levels in the hippocampus or frontal cortex of rats are increased, and wherein a test compounds giving rise to such increase is selected.

6. The method according to claim 5, wherein a test compound is selected if it gives rise to an increase in the extra cellular serotonin levels of 300% or more as measured acutely.

7. The method of claim 5, wherein a test compound is selected if it gives rise to an increase in the extra cellular serotonin levels of 300% or more as measured 3 days after the start of the administration of the test compound.

8. The method according to claim 5, wherein said increase is determined in the hippocampus.

9. The method according to claim 5, wherein said increase is measured in the prefrontal cortex.

10. The method according to claim 1, wherein said allosteric site is the site capable of binding 2-(6-Fluoro-1H-indol-3-ylsulfanyl)-benzyl]-methyl-amine.

11. A method of treating depression which method comprises the administration of a therapeutically effective amount of a compound identified by a method according to claim 1 to a patient in need thereof provided said compound is not [2-(6-Fluoro-1H-indol-3-ylsulfanyl)-benzyl]-methyl-amine.

12. The use of a compound identified by the method according to claim 1 in the manufacture of a medicament for the treatment of depression provided said compound is not [2-(6-Fluoro-1H-indol-3-ylsulfanyl)-benzyl]-methyl-amine.

13. A compound identified by a method according to claim 1 for use as an antidepressant provided said compound is not [2-(6-Fluoro-1H-indol-3-ylsulfanyl) -benzyl] -methyl-amine.

14. A pharmaceutical composition comprising a compound identified by a method according to claim 1 provided said compound is not [2-(6-Fluoro-1H-indol-3-ylsulfanyl)-benzyl]-methyl-amine.

15. A method for promoting the sales of an antidepressant, said method comprising the public distribution of information describing that the effects of said antidepressant is fully or partially attributable to the binding of said antidepressant to an allosteric site on the serotonin transporter to which [2-(6-fluoro-1H-indol-3-ylsulfanyl)-benzyl]-methyl-amine binds.

16. A method for promoting the sales of an antidepressant which binds to the allosteric site of the serotonin transporter capable of binding [2-(6-Fluoro-1H-indol-3-ylsulfanyl)-benzyl]-methyl-amine, said method comprising the public spreading of information describing that the effects of said antidepressant is fully or partially attributable to its binding to an allosteric site.

17. The method according to claim 15 wherein said antidepressant inhibits the serotonin transporter.

18. The method according to claim 16 wherein said antidepressant inhibits the serotonin transporter.
